SEQUENCE LISTING (6 sequences)

SEQ ID NO: 1 - RNA, 159 nt, Homo sapiens
guaaguguagccuagaaauuggggcuggauuugaaaauuagccccaauucugcaauuuucaccgcaauaaaagcuucuccaguuauacauggugauuggucuugaugggcuauuguggacagaggagggugcuagguugggguggacggggccacagcu

SEQ ID NO: 2 - DNA, 159 bp, Homo sapiens
gtaagtgtagcctagaaattggggctggatttgaaaattagccccaattctgcaattttcaccgcaataaaagcttctccagttatacatggtgattggtcttgatgggctattgtggacagaggagggtgctaggttggggtggacggggccacagct

SEQ ID NO: 3 - RNA, 19 nt, Artificial Sequence
  siRNA sense sequence
ggcuauuguggacagagga

SEQ ID NO: 4 - RNA, 19 nt, Artificial Sequence
  siRNA antisense sequence
uccucuguccacaauagcc

SEQ ID NO: 5 - RNA, 19 nt, Artificial Sequence
  siRNA sense sequence
acauggugauuggucuuga

SEQ ID NO: 6 - RNA, 19 nt, Artificial Sequence
  siRNA antisense sequence
ucaagaccaaucaccaugu

(19) United States
(12) Patent Application Publication
(10) Pub. No.: US 2023/0183705 A1
(43) Pub. Date: Jun. 15, 2023

(54) PRODUCTS FOR SUPPRESSING OR REDUCING THE EXPRESSION OR ACTIVITY OF A SNORNA AND USES THEREOF IN THE TREATMENT OF CANCER

(71) Applicants: NINOVAX, PARIS (FR); CENTRE NATIONAL DE LA RECHERCHE SCIENTIFIQUE, PARIS (FR); UNIVERSITY PARIS-SACLAY, GIF-SUR- YVETTE (FR)

(72) Inventors: JEAN-RENÉ MARTIN, ORSAY (FR); JÉRÔME BIGNON, LE VAL SAINT-GERMAIN (FR)

(21) Appl. No.: 17/908,933

(22) PCT Filed: Mar. 4, 2021
(86) PCT No.: PCT/EP2021/055413

§ 371 Date: Sep. 2, 2022

(30) Foreign Application Priority Data
Mar. 4, 2020 (EP) 20305227.9

(51) Int. Cl. C12N 15/113; A61P 35/00; A61K 31/5383; A61K 31/551; A61K 31/519; A61K 38/12; A61K 31/407; A61K 31/122; A61K 31/353; A61K 31/365; A61K 31/475

(52) U.S. Cl. CPC C12N 15/1135; A61K 31/122; A61K 31/353; A61K 31/365; A61K 31/407; A61K 31/475; A61K 31/519; A61K 31/551; A61K 31/5383; A61K 38/12; A61P 35/00; C12N 2310/14; C12N 2310/321; C12N 2310/531; C12N 2310/3231

(57) ABSTRACT

The present invention relates to the field of medicine. It relates more particularly to a product suppressing or reducing the expression or activity of the human small nucleolar RNA (snoRNA) of sequence SEQ ID NO: 1 for use as a medicament. The product of the invention is preferably for use for preventing or treating cancer. The description further relates to vectors, cells, vehicles and compositions capable of delivering and expressing a product suppressing or reducing the expression or activity of the human small nucleolar RNA (snoRNA) of sequence SEQ ID NO: 1, and to uses thereof.

The present invention relates to the field of medicine. It relates more particularly to a product suppressing or reducing the expression or activity of the human small nucleolar RNA (snoRNA) of sequence SEQ ID NO: 1 for use as a medicament. The product of the invention is preferably for use for preventing or treating cancer. The description further relates to vectors, cells, vehicles and compositions capable of delivering and expressing a product suppressing or reducing the expression or activity of the human small nucleolar RNA (snoRNA) of sequence SEQ ID NO: 1, and to uses thereof.

### TECHNOLOGICAL BACKGROUND

Cancer remains a major public health problem worldwide. Cancer occurs when cell division gets out of control and results from impairment of a DNA repair pathway, the transformation of normal genes into oncogenes or the malfunction of tumor supressor genes. Many different forms of cancer exist. The incidence of these cancers varies but it represents the second highest cause of mortality, after heart disease, in most developed countries. Despite the advance in cancer therapy over the last few decades, the medical community still faces with the challenge of treating many types of cancer. Accordingly, there is still a need for a more effective and safe cancer treatment, for example for cancers wherein a dysregulation of ribosome biogenesis is observed. Indeed, abnormal increases in nucleolar size and number caused by dysregulation of ribosome biogenesis has emerged as a hallmark in the majority of spontaneous cancers (Hald et al., 2019).

SnoRNAs are a class of non-coding RNAs that primary accumulate in the nucleoli and consist of 60-300 nucleotides. They are divided into two classes: C/D box snoRNAs and/ H/ACA box snoRNAs. The canonical function of C/D box and H/ACA box snoRNAs are 2′-O-ribose methylation and pseudouridylation of ribosomal RNAs (rRNAs), respectively (cf. FIG. 1). SnoRNAs are involved in various physiological and pathological cellular processes. Emerging evidence suggest that snoRNAs have tumor-suppressive or, on the contrary, oncogenic functions in various cancer types (cf. Liang et al., July 2019, Table 1).

Inventors now herein advantageously identify the human small nucleolar RNA (snoRNA) of sequence SEQ ID NO: 1 (described by inventors in WO2015/067727) as a relevant therapeutic target in the treatment of cancer and provide new products for suppressing or reducing its expression or activity.

### SUMMARY OF THE INVENTION

The present invention provides new therapeutic agents. The description relates to the prophylactic or therapeutic use of products of interest which have been designed by inventors to suppress (“silence”) or reduce the expression or activity of the human small nucleolar RNA (snoRNA) of sequence SEQ ID NO: 1.

A first object herein described by inventors is a product suppressing or reducing the expression or activity of a target RNA sequence, typically of the human snoRNA of sequence SEQ ID NO: 1. Is in particular herein described a product reducing or suppressing the expression or activity of the human snoRNA of sequence SEQ ID NO: 1 for use as a medicament, preferably for use in the prevention or treatment of cancer in a subject.

In a preferred aspect, the product is a nucleic acid molecule, in particular a nucleic acid interfering with the expression or activity of the snoRNA of sequence SEQ ID NO: 1. In a particular aspect, the nucleic acid molecule is a RNA, preferably a snoRNA.

Another object is a DNA sequence coding for a RNA interfering with the expression or activity of the snoRNA of sequence SEQ ID NO: 1.

The description also concerns a vector permitting/allowing the in vitro, ex vivo or in vivo expression of a product according to the invention, typically of a nucleic acid, in particular of a RNA sequence, suppressing or reducing the expression or activity of the human snoRNA of sequence SEQ ID NO: 1.

The description in addition concerns a cell comprising anyone of the products herein described, in particular a cell transformed with a vector according to the invention.

It also concerns a vehicle allowing the transport to a cancerous tissue or cell of a subject of anyone of the products herein described or of a combination thereof, in particular of a product according to the invention for suppressing or reducing the expression or activity of the snoRNA of sequence SEQ ID NO: 1 and/or of a vector according to the invention.

The description further relates to a composition comprising anyone of the products herein described together with a pharmaceutically acceptable support.

Also herein described is the in vivo, in vitro or ex vivo use, in one or the other of the applications described in this text, in particular in the prevention or treatment of cancer in a subject, of anyone of the products of the invention, typically of a vector, a cell, a vehicle, a composition, or any combination thereof.

Another object herein described is a tumor cell, whose genome has been genetically modified to overexpress the snoRNA sequence of SEQ ID NO: 1. Also herein described is a population of cells comprising, or consisting in, such genetically modified tumor cells.

The invention also covers a method, performed in vitro, for screening or identifying a compound/molecule suitable for preventing or treating cancer, and a method, performed in vitro, for evaluating the therapeutic efficacy of a test compound/molecule for preventing or treating cancer, said methods comprising i) exposing to the test compound a tumor cell genetically modified to overexpress the snoRNA sequence of SEQ ID NO:1 or a population of cells comprising such a genetically modified tumor cell, ii) evaluating the effects, if any, of the test compound on the tumor cells, in particular evaluating the therapeutic efficacy of said test compound, the death of tumor cells and/or a stabilization or decrease of tumor cells’ proliferation being correlated with the therapeutic efficacy (anticancer effect) of said test compound. In a particular aspect, the method comprises during step ii) the comparison of the number of living tumor cells in the population of tumor cells which has been exposed to the test compound with the number of living tumor cells in a control population of tumor cells which has not been exposed to the test compound.

These methods for screening, identifying or evaluating a compound/molecule can optionally further comprise a step of administering the in vitro selected compound/molecule in a model animal or in a tumor model cell line and analyzing the effect on the disease progression or tumor cell survival or proliferation.

The description also concerns a method for treating a cancer in a subject, wherein the method comprises a step of administering (a therapeutically efficient amount of) a product as herein described suppressing or reducing the expression or activity of the human snoRNA of sequence SEQ ID NO: 1. It further relates to the use of such a product as herein described for preparing a medicament or pharmaceutical composition for preventing or treating a cancer. In a preferred aspect, the cancer is a cancer wherein a dysregulation of ribosome biogenesis is observed.

The description also concerns kits comprising any combination of the herein described products, typically any combination of nucleic acid(s), vector(s), cell(s), vehicle(s) and/or composition(s).

### DETAILED DESCRIPTION

Inventors have demonstrated that a decrease in the expression of the snoRNA of sequence SEQ ID NO: 1 surprisingly causes a spectacular decrease in tumor cell proliferation. This advantageous therapeutic effect has been demonstrated on five distinct human cancer cell lines (colon cancer, glial cells cancer, lung, breast and leukemia cell lines).

In the context of the present invention SEQ ID NO:1 designates the following human snoRNA sequence:

5′-
GUAAGUGUAGCCUAGAAAUUGGGGCUGGAUUUGAAAAUUAGCCCCAAUUC
UGCAAUUUUCACCGCAAUAAAAGCUUCUCCAGUUAUACAUGGUGAUUGGU
CUUGAUGGGCUAUUGUGGACAGAGGAGGGUGCUAGGUUGGGGUGGACGGG
GCCACAGCU-3′ .

The human snoRNA of sequence SEQ ID NO: 1 is also herein identified as “the snoRNA”.

The human DNA sequence encoding this snoRNA sequence is located on chromosome 11, in position 12822722-12822880 (SEQ ID NO: 2) (Soule et al., Nat. Comm., 2020).

A first object herein described by inventors is an inhibitor of the expression or activity of the human snoRNA of sequence SEQ ID NO: 1. The term “inhibitor” herein designates a product, typically a therapeutic product, which suppresses (i.e., silences or knock-out) or reduces (i.e., decreases or knock-down) the expression or activity of a target RNA sequence which is the human snoRNA of sequence SEQ ID NO: 1 unless otherwise specified.

Is in particular herein described an inhibitor of (i.e., a product suppressing or reducing the expression or activity of) the human snoRNA of sequence SEQ ID NO: 1 for use as a medicament. “Medicament” means a substance possessing preventive or curative properties. In the context of the invention, a medicament is intended to cure, promote cure, relieve or prevent, in a subject, a pathology, abnormality or impairment or a symptom thereof in a subject who can benefit from it.

In the context of the present invention, the subject is an animal regardless of its sex or age, in particular a mammal, preferably a primate or a human being, in particular a subject suffering, at risk of suffering, or suspected to suffer, of a cancer, in particular of a cancer wherein a dysregulation of ribosome biogenesis is observed. In a particular aspect, the subject is a subject with recurrent/relapsed cancer, in particular a subject with recurrent/relapsed cancer wherein a dysregulation of ribosome biogenesis is observed.

In a particular aspect, the product suppressing or reducing the expression or activity (the “inhibitor”) of the human snoRNA of sequence SEQ ID NO: 1 is an isolated (“natural”) or artificially synthesized (“artificial” / “synthetic”) nucleic acid molecule or sequence, preferably a nucleic acid interfering with the snoRNA expression or activity, i.e. a sequence interacting with/binding the snoRNA sequence itself or a sequence regulating the expression or activity of said snoRNA sequence such as for example a promoter, an enhancer or a silencer, and, at least in part as a consequence of said interaction/binding, preventing or limiting the snoRNA expression or activity. The interaction/binding is preferably a specific interaction/binding, i.e., the inhibitor does not interact with/bind a sequence which is not the snoRNA sequence itself or a sequence regulating the expression or activity of said snoRNA sequence.

The expression of SEQ ID NO: 1 can be reduced/decreased by 1 - 100% by administering an inhibitor as herein described. For example, the expression may be reduced by 0.5, 1, 5, 10, 15, 20, 25, 30, 35, 40, 45, 50, 55, 60, 65, 70, 75, 80, 85, 90, 95, or even 99%. The expression may be reduced by any amount (%) within those intervals, such as for example, 2-4, 11-14, 16-19, 21-24, 26-29, 31-34, 36-39, 41-44, 46-49, 51-54, 56-59, 61-64, 66-69, 71-74, 76-79, 81-84, 86-89, 91-94, 96, 97, 98 or 99. The gene expression can be measured by methods known in the arts, such as serial analysis of gene expression (SAGE) and/or RT-qPCR.

In a preferred aspect, the nucleic acid molecule is an antisense nucleic acid or a guide RNA (gRNA) or short guide RNA (sgRNA) of a CRISPR system.

An antisense nucleic acid is typically capable of snoRNA knockdown.

SnoRNA knockdown refers to when expression of a snoRNA is reduced, but not necessarily completely silenced. This is commonly accomplished via RNA interference (RNAi). RNAi generally designates a phenomenon by which dsRNA specifically reduces expression of a target gene at post-translational level. In normal conditions, RNA interference is initiated by double-stranded RNA molecules (dsRNA) of various length, for example ranging from 15 to 30 base pair length. In vivo, dsRNA introduced into a cell is cleaved into a mixture of short dsRNA molecules.

In the context of the present invention, knockdown is accomplished via RNAi by targeting the snoRNA which is then degraded. Short RNAs can be introduced into the cell as either short hairpin RNAs (shRNAs) or small interfering RNA (siRNAs). In mammalian cells, both shRNAs and siRNAs are at least 10, 15 or 20 base pair (bp) long, typically 19, 20, 21, 22, 23, 24 or 25 bp long, and are designed to have complementarity to the target sequence. In the context of the present invention, they are designed to have complementarity to the snoRNA.

shRNAs are double stranded RNAs (dsRNAs) that contain a loop structure, and are processed into siRNA by the host enzyme DICER, an endo-RNase that contains RNase III domains (Bernstein, Caudy et al. 2001). siRNA are dsRNA containing two-nucleotide 3′ end overhangs and 5′-triphosphate extremities (Zamore, Tuschl et al. 2000; Elbashir, Lendeckel et al. 2001; Elbashir, Martinez et al. 2001). After processing, one strand of the siRNA will be loaded into the RISC (RNA-induced silencing complex). The siRNA will bind to its target based on complementarity. If the binding between the siRNA and the snoRNA is perfect, the RISC will cleave the snoRNA. If the binding between the siRNA and the snoRNA is not perfect it will alter snoRNA activity, but no snoRNA cleavage will occur.

In a particular aspect, the antisense nucleic acid molecule interfering with the snoRNA expression or activity is a RNA, typically a short RNA, in particular a short hairpin RNA (shRNA) or a small interfering RNA (siRNA), preferably a shRNA or siRNA.

In the context of the present invention, shRNA and siRNA are designed to have complementarity to the snoRNA of sequence SEQ ID NO: 1. This complementarity involves at least 18 bases, typically between 18 and 25 bases, preferably at least 19 bases, even more preferably at least 22 bases.

In a preferred aspect, the antisense nucleic acid is a small interfering RNA (siRNA) or a short hairpin RNA (shRNA).

A particular antisense nucleic acid is a siRNA or shRNA comprising SEQ ID NO: 3 as a sense sequence and/or SEQ ID NO: 4 as an antisense sequence.

Another particular antisense nucleic acid is a siRNA or shRNA comprising SEQ ID NO: 5 as a sense sequence and/or SEQ ID NO: 6 as an antisense sequence.

In a preferred aspect, the antisense nucleic acid is a siRNA or shRNA comprising SEQ ID NO: 3 as a sense sequence and/or SEQ ID NO: 4 as an antisense sequence.

In another preferred aspect, the antisense nucleic acid comprises SEQ ID NO: 4 or SEQ ID NO: 6.

Preferred shRNAs sequences comprise, or consist in, anyone of a contiguous sequence of 18 to 25 base pairs (bp) binding any part of SEQ ID NO: 2.

Preferred siRNAs sequences comprise, or consist in, anyone of a contiguous sequence of 18 to 25 bp binding any part of SEQ ID NO: 2.

The CRISPR (Clustered Regularly Interspaced Short Palindromic Repeats) system may also advantageously be used in the context of the invention as it is typically capable of snoRNA knockout. Knockout occurs when a double stranded break has been made to the DNA within the snoRNA coding region. This means that there will be no expression of functional snoRNA in the cell.

The double stranded break triggers one of two repair pathways: either Non-Homologous End Joining (NHEJ) or Homology Directed Repair (HDR) (if a repair template is provided). Because NHEJ is an error-prone process, it will often result in the insertion or deletion of nucleotides (called InDel mutations).

The engineered CRISPR system is composed of two elements: a guide RNA (gRNA) and Cas9 nuclease. These components come together to form a ribonucleoprotein (RNP).

The terms guide RNA (gRNA) or short guide RNA (sgRNA) refer within the meaning of the invention to a RNA molecule capable of interacting with a DNA endonuclease such as Cas9 in order to guide it to a target region of the DNA, typically to the target region of the DNA encoding the snoRNA of interest. The specificity of the cut is determined by the gRNA/sgRNA. Each gRNA or sgRNA comprises two regions:
a first region (commonly called the “SDS” region), at the 5′ end of the RNA, which is complementary to the target DNA region and mimics the endogenous CRISPR system crRNA, and
a second region (commonly called the “handle” region), at the 3′ end of the RNA, which mimics the base-pairing interactions between the tracrRNA (trans-activating crRNA) and the endogenous CRISPR system crRNA and has a double-stranded stem-loop structure ending in the 3′ direction with an essentially single-stranded sequence. This second region is essential for the binding of the gRNA or sgRNA to the DNA endonuclease.

The first region of the gRNA or sgRNA (“SDS” region) varies according to the targeted DNA sequence.

The “SDS” region of the RNA, which is complementary to the target DNA region, comprises at least 5 nucleotides, preferably at least 10, 15, 20, 25, 30, 35 or 40 nucleotides, typically between 5 and 40 nucleotides. Preferably, this region has a length of 15, 16, 17, 18, 19, 20, 21, 22, 23, 24, 25, 26, 27, 28, 29 or 30 nucleotides. The second region of the RNA (“handle” region) has a stem-loop (or hairpin) structure. The handle regions of the different RNAs do not depend on the selected DNA target.

According to a particular aspect, the “handle” region comprises, or consists of, a sequence of at least 1 nucleotide, preferably at least 1, 50, 100, 200, 500 and 1000 nucleotides, typically between 1 and 1000 nucleotides. Preferably, this region has a length of 40 to 120 nucleotides.

The overall length of a gRNA or sgRNA is generally from 50 to 1000 nucleotides, preferably from 80 to 200 nucleotides, and more particularly preferably from 90 to 120 nucleotides. According to a particular embodiment, a gRNA or sgRNA as used in the present invention has a length comprised between 95 and 110 nucleotides, for example a length of about 100 or about 110 nucleotides.

The targeted DNA region/portion/sequence can correspond to a portion of non-coding DNA or a portion of coding DNA. In the context of the present invention, the targeted DNA region/portion/sequence comprises all or part of SEQ ID NO: 2. The targeted DNA region/portion/sequence is followed by a protospacer adjacent motif (“PAM”) sequence that is involved in Cas9 binding.

The “SDS” region of a given gRNA or sgRNA is identical (100%) or at least 80% identical, preferably at least 85%, 90%, 95%, 96%, 97%, 98% or 99% identical to the targeted DNA sequence of SEQ ID NO: 2 encoding the snoRNA of SEQ ID NO: 1 and is capable of hybridizing with all or part of the complementary sequence of said sequence, typically with a sequence comprising at least 5 nucleotides, preferably at least 10, 15, 20, 25, 30, 35 or 40 nucleotides, typically between 5 and 40 consecutive nucleotides, preferably a sequence comprising 10, 11, 12, 13, 14, 15, 16, 17, 18, 19, 20, 21, 22, 23, 24, 25, 26, 27, 28, 29 or 30 consecutive nucleotides.

The nucleic acid molecule suppressing or reducing the expression or activity of the human snoRNA of sequence SEQ ID NO: 1 (i.e., “the inhibitor” or “the inhibitory nucleic acid”, in particular the “inhibitory RNA”), typically anyone of the shRNA, siRNA, gRNA and sgRNA molecules described herein above, can be introduced into the subject as a mature RNA molecule, as a precursor thereof, or as a nucleic acid (DNA molecule) encoding said RNA.

Thus, the (mature or precursor) RNA molecule is for example directly introduced into the subject, or is introduced into the subject as a nucleic acid encoding said RNA.

The nucleic acid (DNA or RNA) molecule may also be introduced into the subject via a vehicle (the nucleic acid being located into the vehicle or at the surface thereof) as further explained herein below.

The inhibitory nucleic acid can be an isolated (“natural”), artificially synthesized (“artificial” / “synthetic”) or recombinantly produced nucleic acid molecule or sequence. When natural, the inhibitory nucleic acid may be a mature nucleic acid or a precursor thereof. The inhibitory nucleic acid may also be a modified/an altered nucleic acid that differs from naturally-occurring nucleic acid sequence by the addition, deletion, substitution and/or alteration of one or more nucleotides. Such alterations can include addition of non-nucleotide material, such as to the end of the molecule or to one or more internal nucleotides of the molecule. The nucleic acid molecule can contain modified nucleotides or chemical modifications allowing it, for example, to enhance its stability and/or increase its resistance to nucleases and thus increase its lifespan in the subject. In particular, it can comprise at least one modified or unnatural nucleotide such as, for example, a nucleotide with a modified base such as inosine, methyl-5-deoxycytidine, dimethylamino-5-deoxyuridine, deoxyuridine, diamino-2,6-purine, bromo-5-deoxyuridine or any other modified base allowing hybridization, or acridine substituted nucleotides. The inhibitory nucleic acid used in the context of the invention can also be modified at the level of the internucleotide bond and comprise for example a phosphorothioate, a H-phosphonate or an alkyl-phosphonate bond; or at the level of the backbone and comprise for example an alpha-oligonucleotide or a 2′-O-alkyl ribose, or a peptide nucleic acid (PNA), a Locked Nucleic Acid (LNA), or a Bridge Nucleic Acid (BNA).

The inhibitory nucleic acid may also be modified to have target specificity and/or improved pharmacological properties.

The inhibitory nucleic acid can be prepared by any methods known to the skilled person, such as, for example, chemical synthesis, transcription in vivo or amplification techniques.

An inhibitory antisense nucleic acid for use in the method of the invention can be constructed using chemical synthesis and enzymatic ligation reactions using procedures known in the art. Particularly, antisense RNA can be chemically synthesized, produced by in vitro transcription from linear (e.g., PCR products) or circular templates (e.g., viral or non-viral vectors), or produced by in vivo transcription from viral or non-viral vectors. When the “inhibitor” is introduced into the subject as a sequence encoding an inhibitory RNA, this sequence is placed under the control of an expression promoter.

Another object of the invention is a DNA sequence coding for an “inhibitor” as herein described, typically coding for a shRNA, siRNA, gRNA and/or sgRNA as herein described. Such a DNA sequence is also herein identified as an “inhibitory DNA”.

The invention also concerns any recombinant expression cassette characterized in that it comprises an inhibitory DNA as herein described. The term expression cassette designates a nucleic acid construct/construction comprising a nucleic acid sequence permitting preferably the expression of an “inhibitory RNA” sequence according to the invention and a regulator region, operably linked. The expression “operably linked” indicates that the components are combined so that the expression of the nucleic acid sequence (responsible for the expression of the inhibitory RNA) and/or the targeting of the snoRNA sequence of SEQ ID NO: 1 are under control of the transcriptional promoter. Typically, the promotor sequence is placed upstream of the nucleic acid sequence, at a distance from the latter compatible with expression control. Spacer sequences can be present, between the regulator elements and the coding sequence, as long as they do not impede the expression and/or targeting of the snoRNA sequence of SEQ ID NO: 1.

Another object of the invention concerns any (expression) vector comprising a nucleic acid or a cassette such as previously defined permitting the expression of a inhibitory RNA sequence in a host cell or a host organism. The vector can be a DNA or RNA, circular or otherwise, single or double strand. It is typically a plasmid, phage, viral vector (chosen, for example, from among an adenoviral vector, a retroviral vector, an adenovirus-associated vector, a lentiviral vector, a poxvirus vector, a herpetic vector, etc.), a cosmid or an artificial or synthetic chromosome comprising an expression cassette as defined above. Advantageously, it is a vector capable of transforming a eukaryotic cell, preferably an animal cell, typically a human cell. Such vectors are well known to the person skilled in the art and are particularly described in patent application WO 06/085016 or in the articles by Barton and Medzhitov, 2002; Tiscornia et al., 2004; Xia et al., 2002 et Shen et al., 2003. One preferred vector permitting the expression of an inhibitory RNA according to the invention can be chosen from among, for example, a plasmid, a cosmid, a viral vector or a phage.

An expression cassette or a vector permitting/allowing the in vitro, ex vivo or in vivo expression of a product according to the invention (“an inhibitor”) suppressing or reducing the expression or activity of the human snoRNA of sequence SEQ ID NO: 1, typically of a nucleic acid, in particular of a RNA sequence are thus herein described.

The vectors of the invention can also comprise an origin of replication, a selection gene, a reporter gene and/or a recombination sequence, etc. The vectors can be constructed by standard molecular biology techniques, well known to the person skilled in the art, using, for example, restriction, ligation, cloning, replication, etc. enzymes.

The description in addition concerns a cell comprising anyone of the products herein described, preferably a product for suppressing or reducing the expression or activity of the human snoRNA of sequence SEQ ID NO: 1, in particular a cell comprising an inhibitory nucleic acid sequence, typically an inhibitory RNA sequence according to the invention, or a cell transformed by means of a construct or vector as herein described. The cell is preferably an animal cell, typically a mammalian cell, preferably a human cell.

Herein described inhibitory nucleic acid may be administered in free (naked) form or by the use of delivery systems/vehicles that enhance stability and/or targeting.

A particular aspect of the description thus concerns a vehicle allowing the transport to a target tissue or cell, preferably to a cancerous tissue or cell, of a subject of anyone of the products herein described or of a combination thereof, in particular of an inhibitory product according to the invention for suppressing or reducing the expression or activity of the snoRNA of sequence SEQ ID NO: 1. This vehicle may allow the transport/carry any inhibitory nucleic acid and/or vector as herein described.

The vehicle may be natural, artificial or obtained through recombinant techniques.

The vehicle may be for example a vesicle, in particular a lipidic vesicle such as a liposome [for example a cationic liposome; galactosylated liposome; liposome coated with a ligand allowing it to target a cell type such as an immunoliposome coated with an antibody specific for the target cell (Zheng et al., 2009); a liposome positioned within a nanoparticle formed by polymers (Carmona et al., 2009)] or an exosome; a nanoparticle; a nanocapsule; a microsphere; a bead; a proteinaceous vector (WO 00/53722); a dendrimer; a cyclodextrin; a natural cationic polymer such as chitosan or atelocollagen or a synthetic polymer such as poly(L-lysine), polyethyleneimine (PEI); or multilayer films of polycations and polyanions.

A vehicle such as a dendrimer, a cyclodextrin or a polymer may for example form complexes with an inhibitory nucleic acid of the invention.

The vehicle, for example the vesicle, nanoparticle, nanocapsule or microsphere may be biodegradable, bioerodable or bioadhesive.

In a particular aspect, the vehicle is selected from a lipidic vesicle, an exosome, a nanoparticle, a nanocapsule, a microsphere, a dendrimer and a cyclodextrin.

The vehicle may provide controlled or sustained release of the product.

In a particular aspect, the vehicle is designed to specifically direct/address the inhibitory product to a target cell or tissue, in particular to a target cell or tissue expressing the snoRNA of SEQ ID NO: 1, preferably to a target tumor cell or tissue expressing the snoRNA of SEQ ID NO: 1.

The description further relates to a composition, in particular a pharmaceutical composition, comprising anyone of the products herein described suppressing or reducing the expression or activity of the human snoRNA of sequence SEQ ID NO: 1, together with a pharmaceutically acceptable support, for example an inhibitory RNA sequence, DNA sequence, expression cassette/construct, vector, cell or vehicle as herein described.

The siRNA and shRNA sequences of the invention are very stable and very resistant in vitro. However, in the case of an administration to a subject to be treated, the compositions according to the invention may also advantageously comprise a dietarily-acceptable support or a pharmaceutically-acceptable support.

The expression “dietarily-acceptable support” designates a support/excipient permitting the subject to ingest and digest without risk the composition comprising an “inhibitor” as herein described suppressing or reducing the expression or activity of the human snoRNA of sequence SEQ ID NO: 1, in particular an inhibitory nucleic acid sequence, construct, vector or cell according to the invention, and capable of protecting said inhibitor from any attack, in particular related to food digestion, that could alter it before it produces its therapeutic action.

Examples of dietarily-acceptable supports comprise, for example, sugars, saponins, etc.

The expression “pharmaceutically-acceptable support” designates a support/excipient permitting risk-free administration of the composition comprising an “inhibitor” as herein described suppressing or reducing the expression or activity of the human snoRNA of sequence SEQ ID NO: 1, in particular an inhibitory nucleic acid sequence, construct, vector or cell according to the invention, according to one of the possible administration routes described below.

Possible supports include those suitable for oral, nasal, rectal, topical (including dermal, transdermal, transmucosal, buccal, sublingual and ocular), or parenteral (including cutaneous, subcutaneous, intramuscular, intravenous, intra-arterial and intradermal) administration.

Advantageously, the support of the present composition facilitates penetration of the inhibitory nucleic acid (typically of the inhibitory RNA of interest), the construct or the expression vector according to the invention into cells or tissues, ideally into particular tumor cells or tissues as identified in this text, of the subject being treated, and/or protects the nucleic acid, the construct, the expression vector or cells from any damage that may impair its efficacy.

The choice of carrier as well as the content of active substance in the carrier are generally determined relative to the solubility and chemical properties of the active substance, the mode of administration and the characteristics of the subject, in particular the human being, to be treated. Excipients such as lactose, sodium citrate, calcium carbonate, dicalcium phosphate and disintegrating agents such as starch, alginic acids and certain complex silicates combined with lubricants such as magnesium stearate, sodium lauryl sulfate and talc can be used to prepare tablets. To prepare a lozenge, it is advantageous to use lactose and high molecular weight polyethylene glycols. Aqueous suspensions contain emulsifiers or agents that facilitate suspension. Diluents such as sucrose, ethanol, polyethylene glycol, propylene glycol, glycerol and chloroform or mixtures of these are also usable.

Adjuvants such as aluminum salts (for example aluminum hydroxide, aluminum phosphate, aluminum sulfate), surfactants (such as lysolecithin, pluronic polyols, polyanions, peptides and emulsions), complete and incomplete Freund’s adjuvant, MPL-TDM adjuvant (monophosphoryl lipid A, synthetic trehalose dicorynomycolate), tyrosine, aluminum, saponins such as Stimulon™, and cytokines can also be added to improve the efficiency of the composition.

The compositions for parenteral administration are generally physiologically compatible sterile solutions or suspensions which can optionally be prepared immediately before use from solid or lyophilized form. Adjuvants such as a local anaesthetic, preservative and buffering agents can be dissolved in the vehicle and a surfactant or wetting agent can be included in the composition to facilitate uniform distribution of the active ingredient.

For oral administration, the composition can be formulated into conventional oral dosage forms such as tablets, capsules, powders, granules and liquid preparations such as syrups, elixirs, and concentrated drops. Non-toxic solid supports or diluents may be used which include, for example, pharmaceutical grades of mannitol, lactose, starch, magnesium stearate, sodium saccharine, talcum, cellulose, glucose, sucrose, magnesium, carbonate, and the like. For compressed tablets, binders, which are agents which impart cohesive qualities to powdered materials, are also necessary. For example, starch, gelatine, sugars such as lactose or dextrose, and natural or synthetic gums can be used as binders. Disintegrants are also necessary in the tablets to facilitate break-up of the tablet. Disintegrants include starches, clays, celluloses, algins, gums and crosslinked polymers. Moreover, lubricants and glidants are also included in the tablets to prevent adhesion to the tablet material to surfaces in the manufacturing process and to improve the flow characteristics of the powder material during manufacture. Colloidal silicon dioxide is most commonly used as a glidant and compounds such as talc or stearic acids are most commonly used as lubricants.

For transdermal administration, the composition can be formulated into ointment, cream or gel form and appropriate penetrants or detergents could be used to facilitate permeation, such as dimethyl sulfoxide, dimethyl acetamide and dimethylformamide.

For transmucosal administration, nasal sprays, rectal or vaginal suppositories can be used. The active compound can be incorporated into any of the known suppository bases by methods known in the art. Examples of such bases include cocoa butter, polyethylene glycols (carbowaxes), polyethylene sorbitan monostearate, and mixtures of these with other compatible materials to modify the melting point or dissolution rate.

The invention also concerns the in vivo, in vitro or ex vivo use of anyone of the products herein described, for example of an inhibitory nucleic acid sequence, expression cassette/construct, vector, cell, vehicle or composition as herein described, typically continuously or sequentially, to suppress or reduce the expression, or activity, of the human snoRNA of sequence SEQ ID NO: 1, for example to prepare a composition for preventing or treating a disease, preferably cancer.

In a particular aspect of the description, anyone of the products herein described as capable of suppressing or reducing the expression or activity of the human snoRNA of sequence SEQ ID NO: 1, for example an inhibitory RNA sequence, DNA sequence, expression cassette/construct, vector, cell, vehicle or composition, is herein described as a product for use in the prevention or treatment of a disease, preferably of cancer, in a subject.

In the context of the present description, the terms “cancer” and “tumor” are equivalent. As used herein, they refer to cells possessing characteristics typical of cancer-causing cells, such as uncontrolled proliferation, immortality, metastatic potential, and certain characteristic morphological features. The expressions “cancer cells” or “tumor cells” are equivalent. They are used to identify cells from a tumor (malignant cells), in particular from a primary tumor, circulating tumor cells (in the case of leukaemia for example), cells from a tumor bed, or cells from a metastasis.

The cancer may be any kind of cancer or neoplasia. The cancer is typically selected from a carcinoma, a sarcoma, a lymphoma, a melanoma, a paediatric tumour and a leukaemia tumour.

In a particular aspect, the cancer is a cancer wherein a dysregulation of ribosome biogenesis is observed.

In a particular aspect, the cancer is for example a colon cancer, a rectal cancer, a colorectal cancer, a prostate cancer, a glial cells cancer, a glioblastoma, a neuroblastoma, a breast cancer, a lung cancer [for example a non-small cell lung cancer (NSCLC)], a leukemia (in particular a lymphoid leukemia, an acute lymphoid leukemia or an Hodgkin lymphoma), an oesophagus cancer, a kidney cancer, a thyroid cancer, an osteosarcoma, a gastrointestinal sarcoma (GIST), or a melanoma.

In another particular aspect, the colon cancer is a carcinoma, in particular a colorectal carcinoma. In another particular aspect, the glial cells cancer is a glioblastoma.

In another particular aspect, the breast cells cancer is a breast carcinoma, in particular a breast adenocarcinoma.

In another particular aspect, the cancer is a leukemia, for example a chronic myeloid leukemia.

In a preferred aspect, the cancer is selected from colon cancer, a glial cells cancer, breast cancer, lung cancer and leukemia.

Also herein described is a method for preventing or treating a disease, preferably for preventing or treating cancer, in a subject. The method typically comprises a step of administering a product as herein described for suppressing or reducing the expression or activity of the human snoRNA of sequence SEQ ID NO: 1, typically a therapeutically efficient amount thereof.

Inventors also herein describe the use of such a product for preparing a medicament or pharmaceutical composition for preventing or treating a disease, preferably for preventing or treating cancer, in a subject.

As used herein, the term “treatment” of a disease, in particular of a cancer, refers to any act intended to extend life span of subjects/patients such as therapy and retardation of the disease. The treatment can be designed to eradicate the tumor, to stop the progression of the tumor, to prevent the occurrence of metastasis, to promote the regression of the tumor and/or to prevent tissue invasion of cancer.

The treatment of cancer with a product according to the invention (the “inhibitor”) can be associated with other therapy such as surgery, radiation therapy or chemotherapy.

By a “therapeutically efficient amount” is intended an amount of the therapeutic product of the invention (the “inhibitor”) administered to a subject that is sufficient to constitute a treatment of the disease, in particular of the cancer as defined above, in particular that is sufficient to reduce the symptoms and/or signs of cancer, which include, but are not limited to, weight loss, pain and tumor mass, which is detectable, either clinically as a palpable mass or radiologically through various imaging means.

The inhibitor may be administered as a single dose or in multiple doses, over a period of time appropriate to the disease, in particular to the cancer being treated. The inhibitor may conveniently be administered at appropriate intervals, for example, once a day, twice a day, three times a day, once every second day, once every three days or once every week, over a period of at least 3 months or until the symptoms and signs of the disease, in particular of the cancer, resolved.

Each unit dosage may contain, for example, from 0.01, 0.1 or 0.5 µg of inhibitory nucleic acid /kg of body weight to 1000 mg of inhibitory nucleic acid /kg of body weight, in particular from 0.1 or 0.5 µg of inhibitory nucleic acid /kg of body weight to 200 mg of inhibitory nucleic acid/kg of body weight, from 200 to 1000 mg of inhibitory nucleic acid /kg of body weight, from 500 to 800 mg of inhibitory nucleic acid /kg of body weight, from 0.1, 1, 2, 3, 4, 5, 10 or 15 to 20, 50, 70 or 100 mg of inhibitory nucleic acid /kg of body weight, for example at least about 0.01, 0.1, 0.5 µg of inhibitory nucleic acid /kg of body weight, or at least about 0.1, 1, 2, 3, 4, 5, 10 or 15 mg of inhibitory nucleic acid /kg of body weight.

If the inhibitor is an inhibitory nucleic acid, each unit dosage may preferably contain, for example, from 1 to 50 mg/kg of body weight, particularly from 5 to 20 mg/kg of body weight.

The inhibitor may also be incorporated into a vehicle, for example into an exosome, for its administration/delivery. Different administration routes can be utilized for in vivo administration of an exosomal cargo. For example, intravenous, oral, intraperitoneal, intradermal, and intranasal routes have been successfully used for exosome delivery, as well as intratumoral injection (Das et al., Mol. Pharmaceutics, 2019). For example, a dose of 25, 50 or 100 pmoles (50, 100 or 200 nM) of siRNA can be complexed with exosomes (3 µg per 50 pmoles of siRNA) (Alvarez-Erviti et al., 2011). Then, for example, a dose ranging from 0.1 to 5 mg exosomes /kg of body weight, can be administered to a human subject, for example can be intravenously injected to the subject.

The dosage of the inhibitor administered to the subject in need thereof will depend on the severity of the disease, in particular of the cancer being treated, the particular formulation, and other clinical factors such as weight and the general condition of the subject and route of administration selected by the practitioner.

Typically, products, compositions and medicaments according to the invention may be formulated, as indicated herein above, to release the inhibitor(s) substantially immediately upon administration or at any predetermined time or time period after administration.

In a particular aspect, the product of the invention (the “inhibitor”) is for use in combination with another active agent, in particular with another anticancer agent. The other active agent can be administered simultaneously or consecutively.

In a particular aspect, the anticancer agent is a ribosome synthesis inhibitor such as quarfloxin, CX-5461, BMH-21, Actinomycin D (synonym: Oncostatin K; Actinomycin IV), Thiolutin (synonym: Acetopyrrothin), Resistomycin (synonym: Croceomycin; Geliomycin; Heliomycin; Itamycin), Rubrofusarin (CAS 3567-00-8), Triptolide or Ellipticine.

Another object herein described is a tumor cell, whose genome has been genetically modified to overexpress the snoRNA sequence of SEQ ID NO: 1. Also herein described is a population of cells comprising, or consisting in, such genetically modified tumor cells.

The invention also covers a method, typically performed in vitro, for screening or identifying a compound/molecule suitable for preventing or treating cancer, and a method, typically performed in vitro, for evaluating the therapeutic efficacy of a test compound/molecule for preventing or treating cancer, said methods comprising i) exposing to the test compound a tumor cell genetically modified to overexpress the snoRNA sequence of SEQ ID NO:1 or a population of cells comprising such a genetically modified tumor cell, ii) evaluating the effects, if any, of the test compound on the tumor cells, in particular evaluating the therapeutic efficacy of said test compound, the death of tumor cells and/or a stabilization or decrease of tumor cells’ proliferation being correlated with the therapeutic efficacy (anticancer effect) of said test compound. In a particular aspect, the method comprises during step ii) the comparison of the number of living tumor cells in the population of tumor cells which has been exposed to the test compound with the number of living tumor cells in a control population of tumor cells which has not been exposed to the test compound.

These methods for screening, identifying or evaluating a compound/molecule can optionally further comprise a step of administering the in vitro selected compound/molecule in a model animal or in a tumor model cell line and analyzing the effect on the disease progression or tumor cell survival or proliferation.

The description also concerns a kit comprising any combination of the herein described products, typically any combination of at least two products selected from the nucleic acid(s), vector(s), cell(s), vehicle(s) and/or composition(s) as herein described.

Other aspects and advantages of the present invention will appear upon reading the figures and examples that follow, which should be considered as illustrative and non-limiting.

### Legends of the Figures

FIG. 1: Structural features of C/D box and H/ACA box snoRNAs (adapted from Liang et al., July 2019).

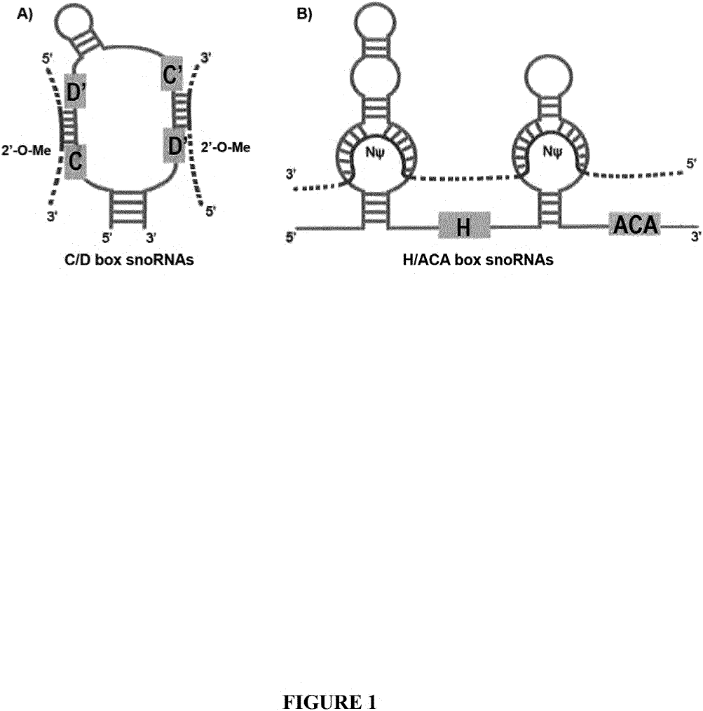

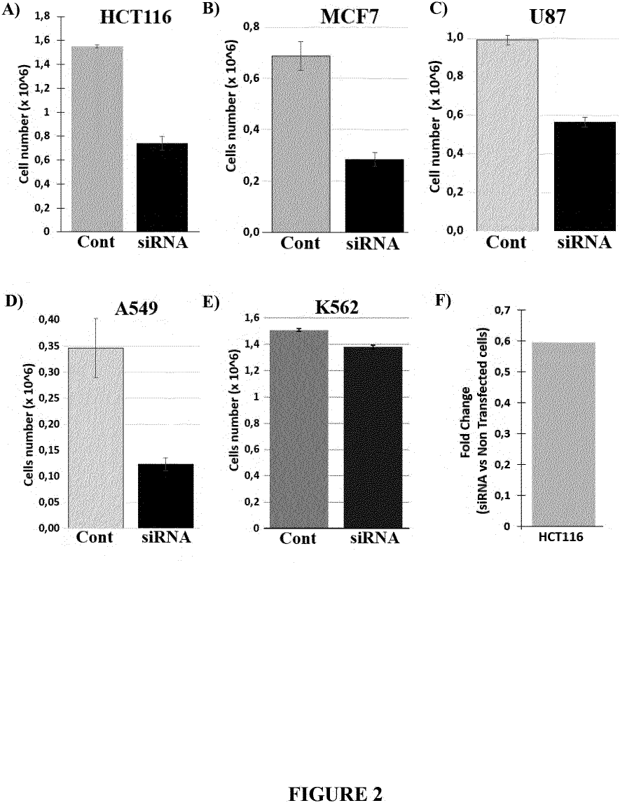

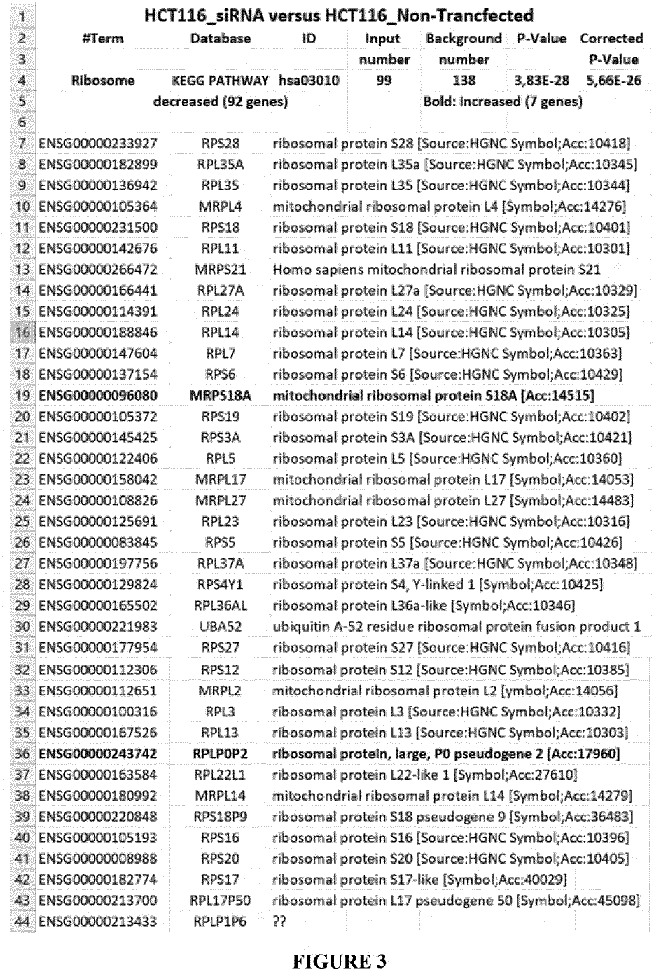

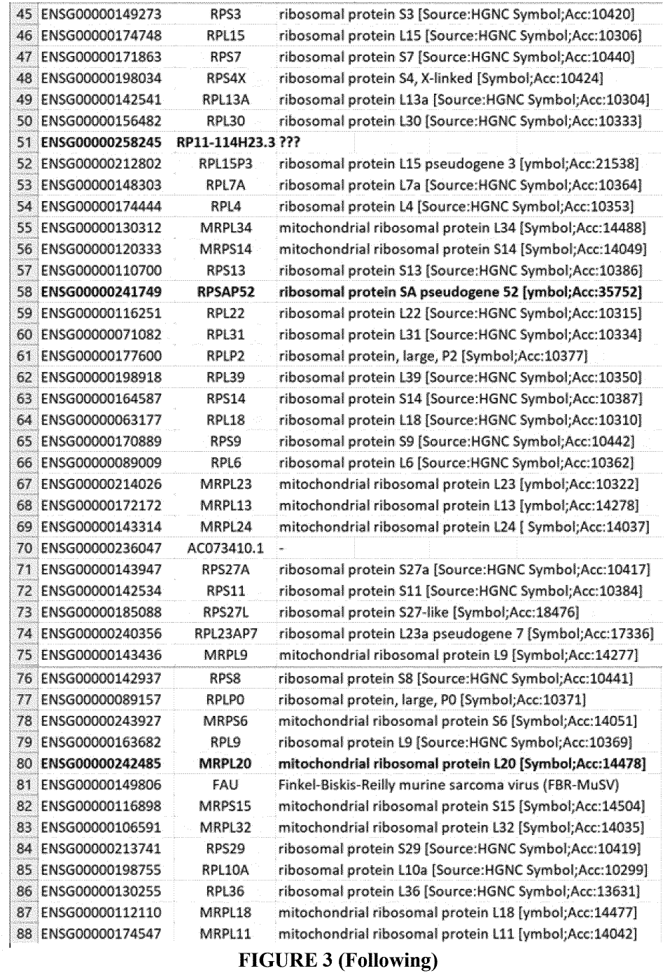

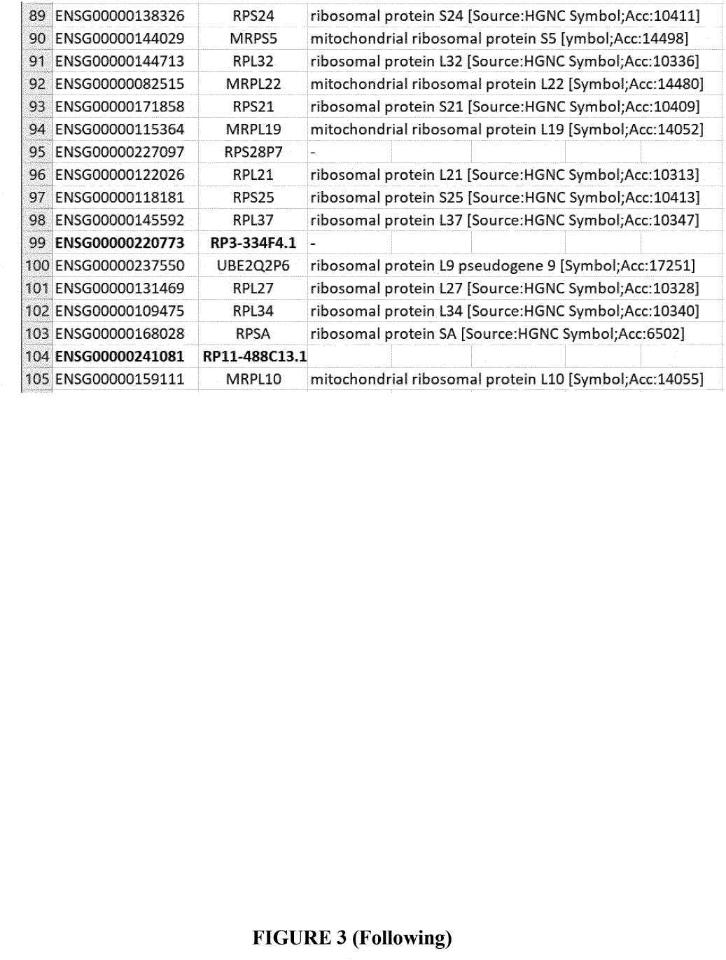

(A) C/D box. Each snoRNAs contains two conserved sequence elements, named box C (RUGAUGA), and box D (CUGA). Box C and box D are close to each other by the base pairing of the 5′ and 3’ termini and fold into a k-turn motif. The antisense elements, upstream of the box D/D′ motifs, are complementary to target RNAs and catalyse site-specific 2′-O-methylation (2’—O—Me) of the nucleotides in target RNAs.

(B) H/ACA box snoRNAs contain evolutionarily conserved structural elements, including box H (ANANNA), box ACA motif, and two pseudouridylation pockets. Pseudouridylation pockets are complementary to the substrate RNAs and responsible for pseudouridylation (NΨ). Dotted line, target RNAs.

FIG. 2: Knock-down of the snoRNA-jouvence on five different human cancer cell lines by treatment with siRNA against the snoRNA-jouvence.

The siRNA treatment consists of three reverse transfections applied at day 0, day 2, day 4, followed by the determination of the number of cells at day 7 for A-B-C-D. For E, a single treatment has been done at day 0, and the number of cells was counted two days later. siRNA was used at a concentration of 10 nM. Five different human cancer cell lines were used. A) HCT116 cells (colon cancer), B) MCF7 cells (breast cancer), C) U87 cells (glioblastoma), D) A549 cells (lung cancer), and E) K562 cells (leukemia). In siRNA-treated cells, inventors observed a strong reduction of the number of cells, by about 50% or more, except in E in which the reduction was about 10% probably due to the fact that only a single treatment has been performed. F displays the histogram of the Fold change for the expression of the snoRNA-jouvence in siRNA-treated HCT116 cells compared to non-treated control HCT116 cells, determined by RT-qPCR (Taqman). In si-RNA treated cells, inventors observed a 40% reduction of snoRNA-jouvence expression.

FIG. 3: List of deregulated genes of the Ribosomes pathways revealed by the KEGG analysis following a Transcriptomic Analysis (RNA-seq) performed on the HCT116 (siRNA treated versus Non-treated cells) (same batch of HCT116 cells as in FIG. 2). Briefly, 99 genes over 138 genes of this pathway are deregulated. Among them, 92 genes are downregulated, while 7 genes (in bold) are upregulated.

### EXPERIMENTAL PART

### Materials and Methods

### Cell Lines and Culture Conditions

All human cancer cell lines were provided from ATCC. HCT116 were cultured in McCoy’s 5A medium (Gibco, Invitrogen, USA), supplemented with 10% Fetal Bovine Serum (FBS) (Biowest, France). MCF7, A549, and K562 cells were cultured in RPMI 1640 + Glutamax medium (Gibco, Invitrogen, USA), supplemented with 10% Fetal Bovine Serum (FBS) (Biowest, France). U87 cells were cultured in Dulbecco’s modified Eagle’s medium (DMEM) (Gibco), supplemented with 10% FBS. Cells were incubated at 37° C. in a humidified atmosphere containing 5% CO₂. The medium was changed every two days, and cells were transferred using 0.05% trypsin/EDTA (Gibco Invitrogen).

### Transfection of siRNA

The effect of the knock-down of the snoRNA-jouvence in HCT116 cells was assessed by the transfection of short interfering RNA (siRNA). First, the cells were seeded in 12-well plates (in triplicates), with the following number of cells per well: HCT116 and MCF7: 3.0×10⁴, K562: 2.5 x10⁴, A549: 2.0x10⁴, and U87: 6.0x10⁴. For the transfection, the silencer selected siRNA (Lock-Nucleotide-Acid siRNA, “LNA-siRNA”) comprising SEQ ID NO: 3 as a sense sequence and SEQ ID NO: 4 as an antisense sequence (ThermoFisher Scientific, USA) was transfected into the cells in a 12-well format, while in parallel, the Control cells were transfected with a non-targeting siRNA control used to demonstrate the specificity of the knock-down (ThermoFisher Scientific, USA, Catalogue number: 4390846). Cell suspensions were directly transfected with the corresponding siRNA (reverse transfection) at a final concentration of 10 nM per well, using the Lipofectamine RNAiMAX transfection reagent (ThermoFisher Scientific, USA). Forty-eight hours after the first siRNA reverse transfection, a forward transfection was performed on the adherent cells (at the same concentration as the first reverse transfection). Again, forty-eight hours after the siRNA forward transfection, a second forward transfection was performed on the adherent cells (at the same concentration). Then, after 72 h (meaning at J7 from the starting point), the medium was changed and cells were counted and compared to the different controls. For the RNA extraction and RNA-seq analysis performed in HCT116 cells, cell pellets were made under the same conditions (at J7). Experiments were performed in triplicates and the specific knock-down of the snoRNA-jouvence was validated by standard RT-qPCR (TaqMan). Experiments have been reproduced with a siRNA comprising SEQ ID NO: 5 as a sense sequence and SEQ ID NO: 6 as an antisense sequence, and similar results have been obtained in HCT116 cells.

### Cell Proliferation

Study was performed in five different human cancer cell lines, HCT116 cells (colon cancer), MCF7 cells (breast cancer), U87 cells (glioblastoma), A549 cells (lung cancer), and K562 cells (leukemia). The proliferation rate of siRNA-treated cells (snoRNA jouvence knock-down) compared to non-treated Control cells was analysed by cell counting at J7. Briefly, the supernatant was harvested and cells washed with PBS 1X. Then, cells were trypsinized with 300 µl of 0.05% trypsin/EDTA (Gibco) per well. Trypsine was inactivated with 700 µL of the corresponding medium per well, and then counted with the VicellXR (Cell viability Analyzer, Beckman Coulter).

### Results

Inventors observed a surprisingly strong reduction of the number of cells in five different human cancer cell lines [HCT116 cells (colon cancer), MCF7 cells (breast cancer), U87 cells (glioblastoma), A549 cells (lung cancer), and K562 cells (leukemia)] associated to the knock-down of the snoRNA-jouvence.

### Transcriptomic Analysis (RNA-seq)

A transcriptomic analysis, performed by inventors on the human colon cancer cell line HCT116 has revealed the dysregulation (decreased or increased expression) of 99 genes of the 138 genes involved in the genesis of ribosome (cf. FIG. 3). This knockdown effect of the snoRNA-jouvence is in perfect agreement with the canonical role of the type H/ACA snoRNA, which are known to be directly involved in the ribosome biogenesis and the spliceosome, two molecular functions crucial and essential for the cell survival. Therefore, this canonical result may be extended and extrapolated to other cancer cell types.

In brief, technically, RNA concentration was measured using Qubit® RNA Assay Kit in Qubit® 2.0 Flurometer (Life Technologies, CA, USA). RNA integrity was assessed using the RNA Nano 6000 Assay Kit of the Bioanalyzer 2100 system (Agilent Technologies, CA, USA). Then, a library was prepared for Transcriptome sequencing. A total amount of 2 µg RNA per sample was used as input material for the RNA sample preparations. Sequencing libraries were generated using NEBNext® Ultra™ RNA Library Prep Kit for Illumina® (NEB, USA). mRNA was purified from total RNA using poly-T oligo-attached magnetic beads. Fragmentation was carried out using divalent cations under elevated temperature in NEBNext First Strand Synthesis Reaction Buffer (5X). First strand cDNA was synthesized using random hexamer primer and M-MuLV Reverse Transcriptase RNase H-). Second strand cDNA synthesis was subsequently performed using DNA Polymerase I and RNase H. Remaining overhangs were converted into blunt ends via exonuclease/polymerase activities. After adenylation of 3’ ends of DNA fragments, NEBNext Adaptor with hairpin loop structure were ligated to prepare for hybridization. In order to select cDNA fragments of preferentially 150~200 bp in length, the library fragments were purified with AMPure XP system (Beckman Coulter, Beverly, USA). Then, 3 µl USER Enzyme (NEB, USA) was used with size-selected, adaptor-ligated cDNA at 37° C. for 15 min followed by 5 min at 95° C. before PCR. Then, PCR was performed with Phusion High-Fidelity DNA polymerase, Universal PCR primers and Index (X) Primer. At last, PCR products were purified (AMPure XP system) and library quality was assessed on the Agilent Bioanalyzer 2100 system. The clusteTring of the index-coded samples was performed on a cBot Cluster Generation System using HiSeq PE Cluster Kit cBot-HS (Illumina) according to the manufacturer’s instructions. After cluster generation, the library preparations were sequenced on an Illumina Hiseq platform and 125 bp/150 bp paired-end reads were generated. Differential expression analysis (DEG) of two conditions/groups (on three biological replicates per condition) was performed using the DESeq R package (1.18.0). DESeq provide statistical routines for determining differential expression in digital gene expression data using a model based on the negative binomial distribution. The resulting P-values were adjusted using the Benjamini and Hochberg’s approach for controlling the false discovery rate. Genes with an adjusted P-value <0.05 found by DESeq were assigned as differentially expressed. KEGG analysis enrichment is a database resource for understanding high-level functions and utilities of the biological system, such as the cell, the organism and the ecosystem, from molecular-level information, especially large-scale molecular datasets generated by genome sequencing and other high-throughput experimental technologies (http://www.genome.jp/kegg/) (Kanehisa, et al., 2017; 2018). Inventors used KOBAS software to test the statistical enrichment of differential expression genes in KEGG pathways. The RNA-seq was performed by Novogene (China).

### REFERENCES

Alvarez-Erviti L, Seow Y, Yin H, Betts C, Lakhal S, Wood MJ. Delivery of siRNA to the mouse brain by systemic injection of targeted exosomes. Nat Biotechnol. 2011, 29: 341-345.
Cui Q, Yang S, Ye P, Tian E, Sun G, Zhou J, Sun G, Liu X, Chen C, Murai K, Zhao C, Azizian KT, Yang L, Warden C, Wu X, D’Apuzzo M, Brown C, Badie B, Peng L, Riggs AD, Rossi JJ, Shi Y. Downregulation of TLX induces TET3 expression and inhibits glioblastoma stem cell self-renewal and tumorigenesis. Nat Commun. 2016, 7:10637.
Das CK, Jena BC, Banerjee I, Das S, Parekh A, Bhutia SK, Mandal M. Exosome as a Novel Shuttle for Delivery of Therapeutics across Biological Barriers. Mol Pharm. 2019, 16(1):24-40.
Elmen J, Lindow M, Schütz S, Lawrence M, Petri A, Obad S, Lindholm M, Hedtjarn M, Hansen HF, Berger U, Gullans S, Kearney P, Sarnow P, Straarup EM, Kauppinen S. LNA-mediated microRNA silencing in non-human primates. Nature. 2008, 452(7189):896-9.
Elmen J, Lindow M, Silahtaroglu A, Bak M, Christensen M, Lind-Thomsen A, Hedtjarn M, Hansen JB, Hansen HF, Straarup EM, McCullagh K, Kearney P, Kauppinen S. Antagonism of microRNA-122 in mice by systemically administered LNA-antimiR leads to up-regulation of a large set of predicted target mRNAs in the liver. Nucleic Acids Res. 2008 Mar;36(4):1153-62.
Hald ØH, Olsen L, Gallo-Oller G, Elfman LHM, Løkke C, Kogner P, Sveinbjärnsson B, Flægstad T, Johnsen JI, Einvik C. Inhibitors of ribosome biogenesis repress the growth of MYCN-amplified neuroblastoma. Oncogene. 2019 Apr;38(15):2800-2813.
Kanehisa, M., Furumichi, M., Tanabe, M., Sato, Y. & Morishima, K. KEGG: new perspectives on genomes, pathways, diseases and drugs. Nucleic Acids Res., 45, D353-D361 (2017).
Kanehisa, M., Sato, Y., Furumichi, M., Morishima, K. & Tanabe, M. New approach for understanding genome variations in KEGG. Nucleic Acids Res., 47, D590-D595 (2018).
Li Y, Yin Z, Fan J, Zhang S, Yang W. The roles of exosomal miRNAs and lncRNAs in lung diseases. Signal Transduct Target Ther. 2019 Nov 13;4:47. doi: 10.1038/s41392-019-0080-7. eCollection 2019. Review.
Lin Y, Lu Y, Li X. Biological characteristics of exosomes and genetically engineered exosomes for the targeted delivery of therapeutic agents. J Drug Target. 2020, (2): 129-141.
Luo J, Xiong Y, Fu PF, Li EC, Qu L, Fan X, Cai ZJ, Lin AF. Exosomal long non-coding RNAs: biological properties and therapeutic potential in cancer treatment. J Zhejiang Univ Sci B. 2019 Jun;20(6):488-495. doi: 10.1631/jzus.B 1900039. Review.
Ma J, Kala S, Yung S, Chan TM, Cao Y, Jiang Y, Liu X, Giorgio S, Peng L, Wong AST. Blocking Stemness and Metastatic Properties of Ovarian Cancer Cells by Targeting p70S6K with Dendrimer Nanovector-Based siRNA Delivery. Mol Ther. 2018, 26(1):70-83.
Pelletier J, Thomas G, Volarevic S. Ribosome biogenesis in cancer: new players and therapeutic avenues. Nat Rev Cancer. 2018 Jan;18(1):51-63.
Petri A, Lindow M, Kauppinen S. MicroRNA silencing in primates: towards development of novel therapeutics. Cancer Res. 2009 Jan 15;69(2):393-5.
Stenvang J, Lindow M, Kauppinen S. Targeting of microRNAs for therapeutics. Biochem Soc Trans. 2008 Dec;36(Pt 6):1197-200.
Soule S, Mellottee L, Arab A, Chen C, Martin JR. Jouvence a small nucleolar RNA required in the gut extends lifespan in Drosophila. Nat Commun 11, 987 (2020). https://doi.org/10.1038/s41467-020-14784-l.
Thorsen SB, Obad S, Jensen NF, Stenvang J, Kauppinen S. The therapeutic potential of microRNAs in cancer. Cancer J. 2012 May-Jun;18(3):275-84.

1-14. (canceled)

15. A nucleic acid for suppressing or reducing the expression or activity of the human small nucleolar RNA (snoRNA) of sequence SEQ ID NO: 1, wherein the nucleic acid is a nucleic acid molecule interacting with the snoRNA of sequence SEQ ID NO: 1 or a sequence regulating the expression or activity of said snoRNA sequence.

16. A method of treating cancer in a subject comprising administering a nucleic acid that interacts with the snoRNA of sequence SEQ ID NO: 1 or a sequence regulating the expression or activity of said snoRNA sequence.

17. The method according to claim 16, wherein the cancer is selected from colon cancer, glial cells cancer, breast cancer, lung cancer and leukemia.

18. The method according to claim 16, wherein the nucleic acid molecule is an antisense nucleic acid or a guide RNA (gRNA) or short guide RNA (sgRNA) of a CRISPR system.

19. The method according to claim 18, wherein the antisense nucleic acid is a small interfering RNA (siRNA) or a short hairpin RNA (shRNA), in particular a siRNA or shRNA comprising SEQ ID NO: 3 as a sense sequence and/or SEQ ID NO: 4 as an antisense sequence or comprising SEQ ID NO: 5 as a sense sequence and/or SEQ ID NO: 6 as an antisense sequence.

20. The method according to claim 18, wherein the gRNA or sgRNA binds to at least 15 consecutive nucleotides of SEQ ID NO: 2.

21. The method according to claim 16, wherein the nucleic acid is administered in combination with another anticancer agent.

22. The method according to claim 21, wherein the anticancer agent is a ribosome synthesis inhibitor selected from quarfloxin, CX-5461, BMH-21, Actinomycin D, Thiolutin, Resistomycin, Rubrofusarin, Triptolide or Ellipticine.

23. The method according to claim 16, wherein the subject is a human being.

24. An expression cassette or a vector comprising the nucleic acid of claim 15, the expression cassette or vector comprising a promotor and/or regulator elements promoting the expression of said nucleic acid in tumor cells.

25. The expression cassette or vector according to claim 24, wherein the vector is a plasmid, a cosmid, a viral vector or a phage.

26. A cell comprising the nucleic acid according to claim 15 or expression cassette or vector comprising said nucleic acid.

27. A vehicle comprising the nucleic acid according to claim 15 and/or an expression cassette or vector comprising said nucleic acid, said vehicle being selected from a lipidic vesicle, an exosome, a nanoparticle, a nanocapsule, a microsphere, a dendrimer and a cyclodextrin.

28. A pharmaceutical composition comprising the nucleic acid according to claim 15, an expression cassette or vector comprising said nucleic acid, a cell comprising said nucleic acid, or a vehicle comprising said nucleic acid or an expression cassette or vector comprising said nucleic acid and a pharmaceutically acceptable support.
